(19) United States
(12) Patent Application Publication
(10) Pub. No.: US 2009/0117087 A1
(43) Pub. Date: May 7, 2009

(54) METHODS AND COMPOSITIONS FOR PRINTING BIOLOGICALLY COMPATIBLE NANOTUBE COMPOSITES OF AUTOLOGOUS TISSUE

(75) Inventors: David Carroll, Winston-Salem, NC (US); William Wagner, Clemmons, NC (US); Nicole Levi, Winston-Salem, NC (US); Louis Argenta, Winston-Salem, NC (US); Michael Morykwas, Winston-Salem, NC (US)

Correspondence Address: MYERS BIGEL SIBLEY & SAJOVEC, PO BOX 37428, RALEIGH, NC, 27627, US

(73) Assignees: Wake Forest University, Winston-Salem, NC (US); Wake Forest University Health Sciences, Winston-Salem, NC (US)

(21) Appl. No.: 12/250,820

(22) Filed: Oct. 14, 2008

Related U.S. Application Data
(63) Continuation-in-part of application No. PCT/US2007/009161, filed on Apr. 13, 2007.
(60) Provisional application No. 60/979,963, filed on Oct. 15, 2007.

(51) Int. Cl. A61K 35/12

(52) U.S. Cl. 424/93.7; 424/572; 977/904; 977/742

(57) ABSTRACT

A method of carrying out an autologous tissue implant in a subject in need thereof is carried out by: (a) forming an autologous tissue implant from autologous cells collected from a subject (e.g., by ink-jet printing, the autologous cells and the scaffold, separately or together), and then (b) implanting the autologous tissue implant in said subject.

### RELATED APPLICATIONS

This application claims the benefit under 35 U.S.C. § 119(e) of U.S. Provisional Patent Application No. 60/979,963, filed Oct. 15, 2007 (attorney docket no. 9151-104Pr);

and also is a Continuation-in-Part and claims the benefit under 35 U.S.C. § 120 of co-pending PCT International Application No. PCT/US2007/009161, filed Apr. 13, 2007 (attorney docket no. 9151-77WO) and published in English under PCT Article 21(2), which claims priority to U.S. Provisional Patent Application No. 60/744,855, filed Apr. 14, 2006;

the entire contents of all of these applications are incorporated by reference herein in their entirety.

### GOVERNMENT FUNDING

This invention was made with Government support under grant number FA9550-04-1-0161 from the Air Force AFOSR. The US Government has certain rights to this invention.

### FIELD OF THE INVENTION

The present invention concerns methods and compositions useful for the production of three-dimensional constructs of viable cells.

### BACKGROUND OF THE INVENTION

Development of three-dimensional tissue scaffolds into which cells may be seeded to generate new tissue is a broad venture that incorporates biomaterial type, strength, and structure tailored for specific cell types. Well-designed scaffolds should consist of biopolymers which may slowly be reabsorbed into the body following implantation, while simultaneously promoting cell adhesion, proliferation, and production of extracellular matrix proteins.¹ Addition of single-wall carbon nanotubes into a polymer system may increase the strength and stiffness of the structure and also offers a means to apply electrical stimulus to cells seeded into the matrix.^{2,2-7} Recent studies indicate that electrically stimulated cells cultured atop carbon nanotubes proliferate more rapidly than on control surfaces and further, secrete their own extracellular matrix proteins following attachment.^{3 8}

There are many means for scaffold development, including lithography,⁹ leaching techniques,¹⁰ hydrogels,^{11,12} electro spinning,^{13,14} and inkjet printing.¹⁵⁻¹⁸ Each method has the ability to produce stable porous scaffolds for infiltration of cells. However, current research indicates that cells proliferate best on nanostructured substrates as compared to smoother surfaces.^{6,7} In this regard, techniques such as electrospinning are promising because they generate biopolymer fibers in the nanometer regime. There are challenges to the utilization of techniques like electrospinning however, such as incompatibility with the formation of fully three dimensional scaffolds with architecture and difficulty with the use of nanocomposites which may be desired for further functionalities.

### SUMMARY OF THE INVENTION

We have developed a composition of printable, biocompatible, “inks” for use in the creation of tissue scaffolds in three dimensions. In general, and in some embodiments, this composition comprises, consists of or consists essentially of a host material (sometimes referred to as a physiologically acceptable polymer) such as; collagen, alginates, fibronectin, elastin, poly(lactide), poly(glycolide), etc., and mixtures or co-polymers, thereof, in some embodiments a bi-phasic dispersant agent such as PEG, and finally a nanophase dispersant. The function of the host is to provide a scaffolding surface for the growth of tissues, the dispersant can be used to mediate solvent drying, or to aid in the dispersion of the nanophase. Finally the nanophase is used to impart functionalities to the scaffolding such as stiffening, strengthening, etc.

A first aspect of the invention is, accordingly, a method for forming an array of viable cells by depositing, spraying, or printing a cellular composition of the cells on a substrate (e.g., under conditions in which at least a portion of the cells remain viable. The substrate employed is a scaffold that comprises, in combination, nanoparticles and a polymer.

A second aspect of the invention is an array (e.g., a tissue scaffold) comprising, in combination,

(a) a scaffold, said scaffold comprising nanoparticles and a polymer; and (b) viable cells deposited (e.g., by printing or ink-jet printing) on the scaffold.

A further aspect of the invention is a liquid composition useful for forming a scaffold for viable cells, comprising (a) nanoparticles; (b) polymer; and (c) solvent.

A further aspect of the present invention is the use of a liquid composition as described herein for carrying out a method as described herein.

In one embodiment, the methods and compositions described above and below are preferably carried out with autologous cells: That is, cells harvested from the same subject that will receive the implant formed by printing of the cells with nanoparticles as described herein.

Thus the present invention provides a method of carrying out an autologous tissue implant in a subject in need thereof, comprising the steps of:

(a) forming an autologous tissue implant from autologous cells collected from a subject (e.g., by ink-jet printing, the autologous cells and the scaffold, separately or together, in any order or in combination, such as by: (i) ink-jet printing the cells on an optionally porous substrate and (ii) ink-jet printing a scaffold for the cells on the optionally porous substrate, the scaffold comprising a physiologically acceptable polymer (and optionally but preferably nanoparticles), and (iii) optionally repeating steps (i) and (ii) to form the autologous tissue implant); and then

(b) optionally applying a cap layer to the implant; and then

(c) optionally repeating steps (b) and (c) (with the same or different autologous cells, nanoparticles, and cap layers) from 1 or 2 to 10, 50, 100 or 1000 times, or more; and then

(d) implanting the autologous tissue implant in the subject.

In some embodiments of the foregoing, the ink jet printing is carried out on an electrospun or electrosprayed substrate (e.g., an inert or biodegradable electrospun or electrsprayed polymer, such as selected from the group consisting of chitosan, collagen, polycitrate, polylactide, chondroitin sulfate and other glycosoaminoglycans or proteoglycans, or combinations thereof, optionally cross-linked after electrospinning with a cross-linking agent such as a carbodiimide, an aldose sugar, D-1-glyceraldehyde, ginipin or glutaraldehyde).

In some embodiments of the foregoing, the cap layer is preformed or inkjet printed thereon.

In some embodiments of the foregoing, the cap layer is preformed (e.g., an electrospun or electrosprayed cap layer (for example, an inert or biodegradable electrospun or electrsprayed polymer, such as selected from the group consisting of chitosan, collagen, polycitrate, polylactide, chondroitin sulfate and other glycosoaminoglycans or proteoglycans, or combinations thereof, optionally cross-linked after electrospinning with a cross-linking agent such as a carbodiimide, an aldose sugar, D-1-glyceraldehyde, etc.)).

In some embodiments of the foregoing, the nanoparticles are antibacterial nanoparticles.

In some embodiments of the foregoing, the nanoparticles are metal nanoparticles (e.g., silver nanoparticles).

In some embodiments of the foregoing, the nanoparticles are electrically conductive.

In some embodiments of the foregoing, the autologous cells comprise skin cells, the subject is afflicted with a wound (e.g., a burn, laceration, crush injury, incision, or combination thereof), and the autologous tissue implant is applied to the wound, optionally followed by treating the wound, the autologous tissue implant, or both the wound and the autologous tissue implant with negative pressure wound therapy.

In some embodiments of the foregoing, the autologous cells comprise smooth muscle cells or endothelial cells, the subject is afflicted with a defective region in a smooth muscle organ wall, and the autologous tissue implant is applied to the defective region.

In some embodiments of the foregoing, the autologous cells are cardiac muscle cells, the subject is afflicted with a defective region in a heart wall, and the autologous tissue implant is applied to the defective region.

In some embodiments of the foregoing, the autologous cells are chondrocytes, the subject is afflicted with a defective region in cartilage, and the autologous tissue implant is applied to the defective region.

In some embodiments of the foregoing, wherein the autologous cells are fat cells, the subject has a region in need of tissue augmentation, and the autologous tissue implant is implanted into the region in need of tissue augmentation.

In some embodiments of the foregoing, wherein the autologous cells comprise skin and fat cells, the subject is afflicted with a wound in need of tissue augmentation, and the autologous tissue implant is applied to the wound, optionally followed by treating the wound, the autologous tissue implant, or both the wound and the autologous tissue implant with negative pressure wound therapy.

A further aspect of the present invention is the use of nanoparticles and/or autologous cells for the manufacture of an autologous tissue implant for carrying out a method as described above.

A further aspect of the present invention is an autologous tissue implant produced by a process as described above.

The present invention is explained in greater detail in the specification set forth below.

### BRIEF DESCRIPTION OF THE DRAWINGS

FIG. 1: A schematic of one embodiment of the invention. Cells are printed together with matrix-scaffold as described above which utilizes nanoparticulates to deliver functionality.

FIG. 2: A schematic of the printed architecture applied to a wound.

FIG. 3: Three sample populations of fibroblasts seeded on printed and cross-linked alginate biopolymer with the addition of silver nanowires (NW) or single-walled carbon nanotubes (SWNT). Each set of two columns represents the total number of cells observed in 10 random fields of view using a 10× objective lens. Glass serves as the control substrate for alginate with and without nanoparticles.

FIG. 4: Three sample populations of keratinocytes seeded on printed and cross-linked chitosan biopolymer with the addition of silver nanowires (NW) or single-walled carbon nanotubes (SWNT). Each set of two columns represents the total number of cells observed in 10 random fields of view using a 40× objective lens. Glass serves as the control substrate for chitosan with and without nanoparticles.

### DETAILED DESCRIPTION OF THE INVENTION

“Nanoparticles” for carrying out the present invention may be in any shape and include rods, ellipsoids, spheroids, tubes (single walled and multi-walled), and complex or combined shapes (e.g., as demonstrated by S. Chen, Z. L. Wang, J. Ballato, S. Foulger, and D. L. Carroll, “Monopod, Bipod, and Tetrapod Gold Nanocrystals”, Journal of the American Chemical Society ja038927. DEC (2003)). The nanoparticles may be composed of any suitable material including carbon (doped and undoped) metals (such as silver, gold, zinc, copper, platinum, iridium, tantalum, etc., including alloys thereof), ceramic (silicon, silica, alumina, calcite, hydroxyapatite, etc.) organic polymers (including stable polymers and bioabsorbable polymers), and composites and mixtures thereof. See, e.g., U.S. Pat. Nos. 6,942,897; 6,929,675; 6,913,825; 6,899,947; 6,888,862; 6,878,445; 6,838,486; 6,294,401; etc. The nanoparticles may be conductive, semiconductive, or nonconductive (insulating). The nanoparticles may be metal nanoparticles formed from metals such as silver, copper, gold, platinum, iridium, and alloys thereof. Carbon nanoparticles (e.g., fullerenes) include nanotubes (including both single-wall and multi-wall nanotubes), buckyballs, fullerenes of other configuration (e.g., ellipsoid), and combinations or mixtures thereof. The nanoparticles may be coupled to (e.g., covalently coupled to) other agents (e.g., proteins, peptides, antibodies) or ligands (e.g., to cell-surface proteins or peptides on the cells being delivered) depending upon the particular application thereof. Diameters of the nanoparticles can be from about 0.1 or 4 nanometers to about 1 micron. Lengths of the nanoparticles can be from 0.8 nm to 100, 200, or 500 microns or more.

“Viable cells” as used herein include prokaryotic and eukaryotic cells such as gram negative and gram positive bacterial cells, yeast cells, plant cells, and animal cells (e.g., reptile, amphibian, avian, mammalian, etc.). Mammalian cells (e.g., human, mouse, rat, monkey, dog, cat, etc.) are in some embodiments preferred. Cells may be of any type, including precursor, progenitor, or “stem” cells, or may be of any suitable tissue (e.g., liver, pancreas, muscle (e.g., smooth muscle, skeletal muscle, cardiac muscle), skin (e.g., epidermal or mesodermal tissue; tissues comprising fibroblasts and/or keratinocytes, etc.), bone (e.g., osteoblast), cartilage (e.g., chondrocytes), tendon, nerve, etc.). In some embodiments the cells are cancer cells (e.g., colon, lung, breast, prostate, brain, liver, or ovarian cancer cells, etc.).

“Polymers” that are used to carry out the present invention may be natural or synthetic and may be bioabsorbable or stable. In general the polymers are preferably physiologically acceptable or biocompatible. Suitable examples include but are not limited to alginate, collagen (including all types of collagen, including Type I, Type III, Type IV, and Type V), fibronectin, polylactide, polyethylene glycol, polycaprolactone, polycolide, polydioxanone, polyacrylates, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof (such as polylactide copolymers including PLGA) See, e.g., U.S. Pat. Nos. 6,991,652 and 6,969,480. Biological materials such as collagen, fibronectin, elastin, etc. may be from any suitable source, e.g., mammalian such as human, bovine, ovine, rabbit, etc.)

“Solvent” as used herein may be any suitable solvent or combination thereof as is known in the art, including but not limited to water, acids such as acetic acid or phosphoric acid, N-methyl-2-pyrrolidone, 2-pyrrolidone, C₂-C₈ aliphatic alcohol, glycerol, tetraglycol, glycerol formal, 2,2-dimethyl-1,3-dioxolone-4-methanol, ethyl acetate, ethyl lactate, ethyl butyrate, dibutyl malonate, tributyl citrate, tri-n-hexyl acetylcitrate, diethyl succinate, diethyl glutarate, diethyl malonate, triethyl citrate, triacetin, tributyrin, diethyl carbonate, propylene carbonate, acetone, methyl ethyl ketone, dimethylacetamide, caprolactam, dimethyl sulfoxide, dimethyl sulfone, caprolactam, N,N-diethyl-m-toluamide, 1-dodecylazacycloheptan-2-one, 1,3-dimethyl-3,4,5,6-tetrahydro-2-(1H)-pyrimidinone, and combinations thereof (see, e.g., U.S. Pat. No. 5,759,563); and/or acetone, benzyl alcohol, benzyl benzoate, N-(betahydromethyl) lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, N-methyl-2-pyrrolidone, esters of caprylic and/or capric acids with glycerol or alkylene glycols, oleic acid, peanut oil, polyethylene glycol, propylene carbonate, 2-pyrrolidone, sesame oil, [+/−]-2,2-dimethyl-1,3-dioxolane-4-methanol, carbitol, triacetin, triethyl citrate, and combinations thereof (see, e.g., U.S. Pat. No. 6,413,536). Preferred solvents include, but are not limited to, water, tetraglycol, polyethylene glycol, acetic acid, dimethyl sulfoxide, C₂-C₈ aliphatic alcohol, vegetable oil such as corn oil, isopropyl myristate, 1-dodecylazacycloheptan-2-one, N-methyl-2-pyrrolidone, and combinations thereof.

“Support” as used herein may be an article of any suitable shape (flat, curved, formed, etc.) and may be made of any suitable material, including metals, glass, ceramics, organic polymers, and composites thereof.

“Negative pressure wound therapy” as used herein is known and describes techniques in which wound healing is facilitated by the application of a vacuum, or negative pressure, to the wound. See, e.g., U.S. Pat. No. 5,645,081 The specific modality of implementation is not critical and any of a variety of techniques can be employed, including but not limited to those described in U.S. Pat. Nos. 7,004,915; 6,951,553; 6,855,135; 6,800,074; 6,695,823; and 6,458,109.

Subjects that may be implanted with constructs or arrays of the present invention include both human subjects and animal subjects (particularly mammalian subjects such as dogs, cats, horses, pigs, sheep, cows, etc.) for veterinary purposes.

The disclosures of all United States patent references cited herein are to be incorporated herein by reference in their entirety.

### 1. Compositions.

As noted above, the present invention provides compositions (sometimes referred to as “ink” compositions) useful for making scaffolds upon which viable cells may be deposited. In general the composition comprises:
(a) nanoparticles (e.g., from 0.1, 0.5 or 1 percent by weight up to 10, 20 or 50 percent by weight);
(b) polymer (e.g., from 1, 2 or 3 percent by weight up to 40, 50 or 60 percent by weight);
(c) a solvent (e.g., from 1 or 5 percent by weight up to 60 or 80 percent by weight, or more); and
(d) optionally, live cells as described herein (e.g., 0, or from 0.01 or 0.1 percent by weight up to 50 or 80 percent by weight of live cells).

Particular examples of polymer and solvent combinations, and advantages and disadvantages thereof, are set forth in Table A below.

In some embodiments the polymer is preferably physiologically acceptable or biocompatible (that is, suitable for implant in a human or animal subject without unduly excessive adverse reaction).

In some embodiments the scaffold is printed separately from the printing or deposition of live cells; in other embodiments the live cells are formulated in and printed with the scaffold ink described herein.

In some embodiments the polymer comprises a single polymer; in other embodiments the polymer comprises a combination of different polymers. Where a combination of different polymers is employed, each polymer in the combination—if charged—can be of the same charge or a different charge.

For some embodiments the composition is preferably in a form suitable for spraying or inkjet printing (discussed further below), and hence preferably has a viscosity of from about 1 or 2 centipoise (and in some embodiments at least 20, 30 or 50 centipoise) up to 60, 80, 100, or 200 centipoise or more. Preferably the nanoparticles in the composition are stably suspended therein (that is, the composition is stable at room temperature without settling of the nanoparticles for at least two weeks, or more preferably at least one month).

### 2. Methods of Making and Using

The compositions described above are applied to a solid support by any suitable means, including spraying or printing. Application may be uniformly or in patterns. In one embodiment, ink-jet printing (e.g., thermal ink-jet printing) is preferred. Thermal ink-jet printing may be carried out with apparatus such as described in U.S. Pat. No. 7,051,654 to Boland, but preferably with the scaffold ink compositions described herein.

The compositions may be applied in a single layer or multiple layers, depending upon the particular end structure or array being produced. Such application forms a “substrate” or “scaffold” on the solid support to which cells may then be applied. The scaffold so formed generally comprises, in combination, nanoparticles (e.g., from 0.01, 0.1, or 1 or 5 to 10, 20 or 50 percent by weight of said scaffold) and a polymer (e.g., from 99 or 95 to 50, 40 or 20 percent by weight of said scaffold).

Once applied the compositions can, if necessary and/or desired be crosslinked by any suitable technique (including chemical, pH, enzymatic, thermal, and light (particularly UV) cross-linking, and combinations thereof.).

Cells are then applied to the scaffold. As with application of the polymer/nanoparticle compositions to the support, the cells may be applied by any suitable means, such as spraying or printing, with ink-jet printing being (in one embodiment) preferred. The cells may be applied as a single application or multiple applications (uniformly or in patterns) to create three dimensional arrays. In some embodiments cells may be sandwiched between multiple layers of nanotube/polymer scaffold layers. Indeed, multiple layers (e.g., 3, 4, 5, 6, 10, 20, 30 or more) of scaffold and cells, in any order or combination, may be carried out to produce the desired structures or arrays such as three-dimensional, contoured, or shaped arrays.

Methods and compositions for forming three-dimensional structures by deposition of viable cells are described in W. Warren et al., U.S. Pat. No. 6,986,739 (Sciperio Inc.). Methods and compositions for the ink-jet printing of viable cells are described in T. Boland et al., U.S. Pat. No. 7,051,654.

In some embodiments, the polymers within the scaffold are cross-linked after they are ink-jet printed. Such cross-linking can be carried out by any suitable technique, such as separately applying (e.g., by ink-jet printing through a different orifice) a cross-linking agent (e.g., a carbodiimide, an aldose sugar, D-1-glyceraldehyde, ginipin, etc.) onto the scaffold, by utilizing polymers that are cross-linked upon exposure to light (e.g., UV light) or heat, etc. An advantage of cross-linking is, in some embodiments, to maintain or enhance the physical integrity of the scaffold.

When desired, the array or scaffold can be washed or rinsed one or more times (e.g., with sterile physiological saline solution, a water/ethanol wash solution) to remove excess solvents therefrom, prior to or after cell deposition and/or implantation).

Once the arrays are formed by the methods described above, the arrays or constructs may be cultured further in vitro in accordance with known techniques to grow the cells (e.g., for subsequent implantation as a prosthesis or the like in a subject, or for the commercial production of a desired compound such as naturally occurring or transgenic protein or peptide from the cells in a fermentation process).

In some embodiments, the growth or proliferation of the viable cells can be enhanced while they are growing in vitro by subjecting the viable cells to an electric field or current sufficient to enhance the proliferation thereof of said viable cells. The electrical field or current may be achieved by any suitable means, such as by connecting the scaffold (directly or indirectly) to a power supply, and/or connecting culture media in which the cells are cultured to a power supply.

TABLE A
BIODEGRADABLE POLYMERS AND CORRESPONDING SOLVENTS.

| | | Thermal &Mechanical Properties | | | | | | |
|---|---|---|---|---|---|---|---|---|
| | | | Glass | | | | | |
| | | Melting | Transi- | Approxi- | Approx. | Degra- | | |
| | | Point | tion | mate | Degradation | dation | | |
| Polymer | Solvent | (° C.) | (° C.) | Strength | Time (weeks) | Product | Advantages | Disadvantages |
| Poly | HFP | 225-230 | 35-40 | 7.0 GPa | 2-4 | Glycolic | Have better than average | Low solubility in |
| (glycolic | | | | (Modulus) | | acid | tissue biocompatibility | organic solvents |
| acid) | | | | | | | Reproducible mechanical | |
| (PGA) | | | | | | | properties | |
| | | | | | | | Hydrophilic | |
| Poly | 1 .HFP | | 60-65 | 2.7 GPa | 30-50 | l-lactic | High solubility in organic | Very hydrophobic |
| (lactic | 2. Chloroform | | | (Modulus) | | acid | solvents | |
| acid) | 3. DCM/DMF | | | | | | | |
| (PLA) | (70:30) | | | | | | | |
| Poly (d,l- | 1. HFP | Amorphous | 45-60 | 2.0 GPa | 4-24 | d,l-lactic | Controllable biodegradation | Systemic or local |
| lactic-co- | 2. Chloroform | | | (Modulus) | (Based on the | acid and | rate and mechanical | reactions due |
| glycolic | 3. DMF | | | | ratio of PLA to | glycolic | properties by varying the | to acidic |
| acid) | 4. THF/DMF | | | | PGA) | acid | ratio of PLA to PGA | degradation |
| (PLGA) | (1:1) | | | | | | | products |
| Poly | 1. HFP | 58-63 | −65-60 | 0.4 GPa | >56 | Caproic | Relatively inexpensive | Too slow |
| (capro- | 2. Chloroform | | | (Modulus) | | acid | Good mechanical properties | degradation |
| lactone) | 3. Chloroform/ | | | | | | (modulus and elasticity) | rate for |
| (PCL) * | methanol | | | | | | Nontoxic and tissue | some |
| | (1:1) | | | | | | compatible polymer | applications |
| Poly | HFP | — | −10-0 | — | 4-20 | — | A biodegradable polymer | exhibits some |
| (dioxanone) | | | | | | | that is, particularly used | shape memory |
| (PDO) | | | | | | | as sutures. | properties |
| | | | | | | | Good flexibility | |
| | | | | | | | Moderate degradatuion rate | |
| Poly | — | — | — | 2 to 30 | Depends on the | Fumaric | An important biodegradable | achieving high |
| (propylene | | | | MPa | formulation, | acid, | and cross-linkable polymer | molecular weight |
| fumarate) | | | | (compressive | several | propylene | designed for bone-tissue- | PPF is difficult |
| (PPF) * | | | | strength | months in vitro | glycol | engineering applications | |
| | | | | | | and poly | | |
| | | | | | | (acrylic | | |
| | | | | | | acid-co | | |
| | | | | | | fumaric | | |
| | | | | | | acid) | | |
| Polyan- | chlorinated | 46-69 | — | 0.045 | Depends on the | Dicar- | A well-defined polymer | hydrolytic |
| hydrides | hydrocarbons | | | (Modulus) | different | boxylic | structure with controlled | instability; low |
| | | | | | polymer types | acids | molecular weight and | mechanical |
| | | | | | | | degrade hydrolytically at a | strength and film |
| | | | | | | | predictable rate. | or fiber forming |
| | | | | | | | | properties |
| Polycar- | Weld-On 4 | 135 | 55-69 | 1.6-2.2 | Very slow | carbondi- | Biocompatible and promotes | Problems of |
| bonate* | (water thin) | | | (Modulus) | Degradation | oxide | bone growth | acid bursting |
| | | | | | (In vitro) | and | | while the |
| | | | | | | alcohols | | polymer degrades |
| Poly | Chloroform | — | 35-95 | 0.85-1.15 | 2-15 | Carboxylic | Mechanical properties of the | Hydrophobic |
| (ortho- | | | | (Modulus) | | acids | polymer can be controlled. | materials |
| esters) | | | | | | | Suitable for orthopedic | |
| (POE) * | | | | | | | applications | |
| Poly- | 1. DMF | — | — | 8 to 40 | 4-8 | Lysine, | Excellent mechanical | Toxicity of |
| urethane | 2 THF/DMF | | | MPa | | glycolic | properties and good | degradation |
| | (1:1) | | | tensile | | and caproic | biocompatibility | product |
| | | | | strength | | acids | | |

*Polymers used for orthopedic application from the literature.
HFP: Hexafluoro-2-propano
DMF: Dimethyl formamide
DCM: Dichloromethane
THF: Tetrahydrofuran.

### 3. Applications.

By making possible the printing of cell scaffolds with functional characteristics that can be enhanced, modified or adjusted in a variety of different ways (depending on, among other things, the selection of nanoparticles used), the present invention has a number of applications. Particular applications include, but are not limited to, the following:

A. Electrically conductive scaffolds. By including electrically conductive nanoparticles, the scaffolds can be operatively associated with a current source (such as a battery or voltage regulator) and used to electrically stimulate cells thereon (e.g., muscle cells, nerve cells, skin cells, or any other cell type for which electrical stimulation stimulates growth or enhances proliferation thereof). Particular electrically conductive nanoparticles include, but are not limited to, metal and carbon nanoparticles and nanotubes, including nanowires. Such scaffolds can also be used for applying heat to the scaffolding.

B. Stiffened scaffolds. By including nanoparticles in the scaffold in an appropriate amount (e.g., from 0.001 or 0.01 percent by weight, Up to 10 or 20 percent by weight of the ink composition), the elastic modulus of the scaffold can be increased by at least 20 or 50 percent, up to 200 or 500 percent or more, as compared to a scaffold of the same configuration and composition without nanoparticles.

C. Patterned scaffolds. By including nanoparticles in the scaffold in an appropriate amount (e.g., from 0.001 or 0.01 percent by weight, up to 10 or 20 percent by weight of the ink composition), cell scaffolds with improved definition of topographical features (such as lines, ridges, wells, vias, composite shapes, etc.) are obtained. For such features, aspect ratios (A/B) of topographical features on the printed scaffold (which may be printed as a single layer or multiple layers as described above) are in some embodiments preferably at least 1, 2, or 3 (where A is the heighth (or depth) and B is the width of the topographical feature, when the topographical feature is measured in cross-section.

D. Contrast agents. Nanoparticles used to carry out the present invention can comprise or contain a contrast or imaging agent to provide detectability of the scaffold in an imaging system such as NMR, X-ray, or the like. Such contrast or imaging agents can comprise Gd complexes, metals such as Fe, and Fe₃O₄, encapsulated contrast agents such as fullerene and encapsulated Gd complexes. See, e.g., U.S. Pat. No. 6,797,380.

E. Antimicrobial nanoparticles. Nanoparticles used to carry out the present invention can comprise or contain an antimicrobial (e.g., antibacterial) agent, such as when the scaffolds are used as a tissue implant scaffold to grow cells for tissue implantation. Antimicrobial metal (including metal alloy) particles can comprise any suitable metal materials (e.g., silver) or bi-, tri- or multicomponent or alloyed metals, typically of a size of from 2 nm to 1000 nm).

F. Active agents. Nanoparticles can be formed of a polymer such as a biodegradable polymer (e.g., PLGA) that contain an active agent to be released into the scaffold. Any suitable active agent beneficial to the cells on the scaffold (or tissue surrounding a region into which the scaffold is implanted) including but not limited to, protein growth factors, cytokines, antibodies, nucleic acids, carbohydrates, antibiotics, etc. See, e.g., PCT Application WO 2006/099333 to Atala et al.

F. Free radical scavengers. Nanoparticles comprised, consisting of, or consisting essentially of a free-radical scavenger can be utilized to produce a scaffold that scavenges such free radicals and reduces their deleterious effects on cells grown thereon. Examples include, but are not limited to, fullerene and transition metal oxides.

H. Others. Other applications of the present invention include quantum dot nanoparticles (e.g., CdSe QD from Evident Technologies) for tracking of targeted or tagged agents within the scaffold, transition metal oxides for catalytic crosslinking. etc.

### 4. Autologous Cells and Implants.

In some embodiments of the invention as noted above, the cells can be autologous cells. Autologous cells can be collected from subjects, processed, printed with nanoparticles as described herein, and prepared for administration back to the subject by any suitable technique, and indeed numerous methods of preparing autologous tissue implants are known which can be facilitated or enhanced by the methods of the present invention.

For example, Stone et al., Composite Collagen Material and Method of Forming Same, U.S. Pat. No. 7,252,832 (Biomet Sports Medicine) describe a felt for repairing cartilage, ligament, or tendon soft tissue defects, comprising: (a) a membranous collagen substrate; and (b) a bioresorbable material felted onto the collagen substrate; wherein the bioresorbable material is selected from a synthetic polymer, a natural polymer, a polysaccharide, or combinations thereof. The synthetic polymer is in some embodiments selected from the group consisting of: polymers and co-polymers of glycolic acid, L-lactic acid, D-lactic acid, urethane urea, trimethylene carbonate, dioxanone, caprolactone, hydroxybutyrate, orthoesters, orthocarbonates, aminocarbonates, and physical combinations thereof; the natural polymer is in some embodiments selected from the group consisting of: elastin, silk, fibrin, fibrinogen, and mixtures thereof; and the polysaccharide is selected from the group consisting of: hyaluronic acid, chitin, chitosan, alginate, carboxymethylcellulose, and mixtures thereof. The felt may further comprise nutrient factors, growth factors, antimicrobials, anti-inflammatory agents, blood products, autologous differentiated or undifferentiated stem cells, and mixtures thereof. Such a felt can be produced by printing a scaffold with nanoparticles as described herein.

Lee, Methods and Compositions for Correction of Cardiac Conduction Disturbances, U.S. Pat. No. 7,252,819 (University of California) describes a process of establishing an electrical connection between a recombinant mammalian cell and a myocardial cell, the method comprising: contacting a myocardial cell of a subject with a recombinant mammalian cell genetically modified to express a recombinant connexin 43 protein, wherein the recombinant cell is a myoblast cell or a cardiomyocyte, wherein the recombinant cell is autologous or allogeneic to the subject, and wherein the contacting is performed by injection into cardiac tissue of the subject or is performed by cardiovascular infusion into the subject and in a manner sufficient to provide for production of an electrical connection between the myocardial cell and the recombinant cell; wherein an electrical connection between the recombinant cell and the myocardial cell is established. Where autologous cells are used, they can be printed together with nanoparticles as used herein, or on a nanoparticle-containing scaffold, to facilitate the contacting by preparing a tissue implant rather than simply injecting cells.

Noval et al., Artificial Dermis and Production Method Therefor, U.S. Pat. No. 7,244,552 (CIEMAT), describes an artificial dermis comprising a gel of clotted human plasma, platelets and cultivated dermal fibroblasts, wherein fibrinogen from the plasma is at a final concentration in the gel of about 0.4 to about 2.0 mg/ml. The fibroblasts may be autologous fibroblasts and the dermis may further comprise autologous keratinocytes. An artificial skin is described in which the artificial dermis is combined with a stratified epithelium. Such artificial dermis and skin may be produced by printing the autologous cells together with a scaffold, preferably containing nanoparticles, as described herein.

Cohen et al., Tissue Engineered Biografts for Repair of Damaged Myocardium, U.S. Pat. No. 7,214,371, describes a method for repairing a damaged myocardium in a mammal, comprising: a) providing a three-dimensional porous polysaccharide matrix; b) introducing mammalian cells (e.g., autologous cells) into the matrix; c) growing the cells in the matrix in vitro, until a tissue-engineered biograft is formed, comprising a contracting tissue; and d) transplanting the tissue-engineered biograft onto myocardial tissue or myocardial scar tissue of the mammal, optionally previously removing scar or dead tissue from the site of implantation; and wherein the polysaccharide matrix may further comprise controlled-release polymeric microspheres, the microspheres being capable of releasing soluble angiogenic growth factors in a controlled manner. Such a biograft can be formed by printing of autologous cells on or with a scaffold, preferably having nanoparticles as described herein.

Kusanagi et al, Method for Treatment and Repair of Meniscal Injuries, U.S. Pat. No. 7,157,428 (Histogenics) describes a method for repair of meniscal injury, lesion or tear by introducing into a site of the meniscus injury, lesion or tear an adhesive rapidly gelling biodegradable derivatized collagen-PEG hydrogel complex wherein the derivatized collagen is alkylated Type I collagen, and wherein the collagen-PEG hydrogel complex introduced into the site of the meniscus injury, lesion or tear further comprises autologous cells. Such a complex can be produced by printing of the autologous cells with a scaffold and/or nanoparticles as described herein.

Hoemann et al., Composition and Method for the Repair and Regeneration of Cartilage and Other Tissues, U.S. Pat. No. 7,148,209 (Ecole Polytechnique), describe a method for repair and/or regeneration in cartilaginous tissue comprising administering at a site of the cartilaginous tissue in need of repair an effective amount of a polymer composition comprising: a solution of a polymer; and blood (e.g., autologous blood cells), wherein the polymer includes at least one selected from the group consisting of a polysaccharide, a protein, and a polyamino acid, and further wherein when the polymer is combined with blood the polymer composition is converted into a non-liquid state in time or upon heating such that the polymer compositions when placed at the site in need of repair, the polymer composition will adhere to the site in need of repair to effect reconstruction or bulking of the tissue and/or regeneration thereof. Such a polymer composition can be produced by printing of autologous blood cells with a scaffold and/or nanoparticles as described herein.

Anderson et al., Prosthetic Grafts, U.S. Pat. No. 7,147,846 (Zimmer Orthobiologics Inc.) describes method for producing a prosthetic graft, comprising: applying one or more adherent cells (e.g., autologous cells) to a porous prosthetic implant for containing blood in vivo, wherein the prosthetic implant has an outer surface that is not in contact with blood flow in vivo and an inner surface that is in contact with blood flow in vivo, the inner surface defining an interior space for containment of blood flow; wherein the adherent cells are applied to the outer surface, and not to the inner surface, of the porous prosthetic implant; and wherein the adherent cells are transfected with at least one recombinant nucleic acid molecule operatively linked to a transcription control sequence, the recombinant nucleic acid molecule encoding a protein that enhances patency of the prosthetic implant; and incubating the implant ex vivo under conditions sufficient to allow the adherence of the adherent cells to the outer surface of the implant. Such an implant or graft can be produced by printing of the autologous cells together with a scaffold and/or nanoparticles as described herein.

Keller et al., Method of Using Autologous Fibroblasts to Promote Healing of Wounds and Fistulas, U.S. Pat. No. 7,115,274 (Isolagen Technologies Inc.) describes a method of promoting healing of a fistula in an animal, wherein the fistula has a fistula tract opening and is susceptible to healing upon administration of autologous fibroblasts, which method comprises: (a) obtaining autologous fibroblasts, and (b) administering the autologous fibroblasts in the form of an injection into the fistula tract opening, wherein the autologous fibroblasts promote healing of the fistula. Such a method can be carried out by printing the autologous fibroblasts together with nanoparticles as described herein to produce a graft and implanting the graft rather than simply administering the fibroblasts in the form of an injection.

Freyman, Selected Cell Delivery for Heart Failure, U.S. Pat. No. 7,097,833 (Boston Scientific) describes a method of increasing blood flow to tissue in a subject in need thereof which comprises: a) isolating autologous mononuclear cells from cardiac muscle from the subject; b) selecting from the isolated autologous mononuclear cells of step (a) lineage negative (Lin⁻) mononuclear cells; and c) transplanting locally into or adjacent to the tissue an effective amount of the Lin⁻autologous mononuclear cells, resulting in formation of new blood vessels in the tissue and formation of new tissue, thereby increasing blood flow to the tissue. Such a method can be carried out by printing the autologous cells together with a scaffold and/or nanoparticles as described herein to produce an implant for the transplant thereof.

Soykan et al., Method and System for Myocardial Infraction Repair, U.S. Pat. No. 7,031,775 (Medtronic) describes a method of repairing the myocardium of a patient,

the method comprising: (a) providing an implantable system comprising: (i) a cell repopulation source comprising genetic material, undifferentiated autologous contractile cells, or a combination thereof, capable of forming new contractile tissue in and/or near an infarct zone of a patient's myocardium; and (ii) an electrical stimulation device for electrically stimulating the new contractile tissue in and/or near the infarct zone of the patient's myocardium; (b) implanting the cell repopulation source into and/or near the infarct zone of the myocardium of a patient; (c) allowing sufficient time for new contractile tissue to form from the cell repopulation source; and (d) electrically stimulating the new contractile tissue. The implantable system can be produced by printing the autologous cells together with a scaffold and/or nanoparticles as described herein.

Hunziker et al., Keratinocyte Culture and Uses Thereof; U.S. Pat. No. 7,014,849 (DFB Pharmaceuticals) describes a method for the treatment of a skin defect comprising (a) culturing an intact hair follicle of an anagenic hair to obtain outer root sheath cells; (b) culturing the outer root sheath cells to obtain keratinocyte precursor cells; (c) preparing an epidermal or dermal equivalent comprising the keratinocyte precursor cells; and (d) applying a portion of the epidermal or complex equivalent to the defect. Preferably the outer root sheath cells are autologous cells obtained from an individual who will subsequently undergo treatment for a skin defect. Such an epidermal or dermal equivalent can be produced by printing of the cells together with a scaffold and/or nanoparticles as described herein.

F. Wood and M. Stoner, US Patent Application Publication No. 2002/0106353, describes methods and apparatus for collecting cells from a donor, dispersing those cells in a solution, and administering the cells to a recipient's graft site. Such cells can be printed with a scaffold and/or nanoparticles as described herein.

Other methods and compositions that can be facilitated by the present invention include but are not limited to those described in U.S. Pat. Nos. 7,048,750 and 7,015,198.

The disclosures of all US patent references cited herein are to be incorporated by reference herein in their entirety.

The present invention is explained in greater detail in the following non-limiting Examples.

### EXPERIMENTAL

In this work we demonstrate a unique compatibility between biopolymer/nanotube composites and thermal inkjet printing that allows for the development of ideal fibrous scaffolds similar in nature to both electrospun material and native tissues. Further such techniques can be used in conjunction with electrospun and natural materials.

Common biomaterials for scaffold development, which we have used here, include alginates, collagen I, fibronectin, and polylactic co-glycolic acid (PLGA) variations. Collagen I and fibronectin are natural biopolymers found in vivo and alginates have been shown to act as viable artificial replacements similar to glycoaminoglycosans which naturally occur in the body. PLGA is a material used in sutures and as additional material in tissue scaffolds, which hydrolyses into glycolic and lactic acids which are reabsorbed by the body. Collagen and other extracellular matrix proteins are typically reincorporated into the tissues following implantation. Likewise, a variety of cell types are known to have increased proliferation on nanofibrous materials such as collagen fibrils or carbon nanotubes.^{3,7,19} Single-wall carbon nanotubes (SWNT) have been shown to act as a viable matrices which do not illicit immune response and are cleared from the body over time.²⁰⁻²⁴ Ideally, incorporation of such nanostructuring into three dimensional biomaterials could provide an added functionality to the scaffold allowing for the potential of creating fully filled organs; one of the primary goals of regenerative medicine.

In our approach, the factors that influence a ‘bio-ink’ and can lead to clogging of printheads include viscosity and concentration; also the solvents for the polymers and nanotubes must be compatible with one another and with the printhead.²⁵ As cited in the literature typical limits for viscosity of print solutions are about 20cP.²⁶ However, we have found that, for solutions with PLGA, viscosities above 20 or 30 cP (e.g., up to 100 cP), allow for fine structure printing, see Table 1. Otherwise, more generally, preferred viscosity ranges for the compositions are from 0.01 cP to 100.

TABLE 1
Viscosities for biopolymer/carbon nanotube composites
using a cone on plate viscometer.¹

| | | Polymer | Nanotube | |
|---|---|---|---|---|
| | | Concen- | Concen- | Vis- |
| | | tration | tration | cosity |
| Polymer | Solvent | (mg/ml) | (mg/ml) | (cP) |
| | tetraglycol | n/a | 0 | 15.4 |
| | tetraglycol | n/a | 0.1 | 17.3 |
| | PEG | 100 | 0.01 | 3.36 |
| collagen | 2% acetic acid in water | 1 | 0 | 6.07 |
| collagen | 2% acetic acids in water | 0.5 | 0.005 | 5.25 |
| | and PEG | | | |
| fibronectin | water and tetraglycol | 0.05% | 0.05 | 3.76 |
| *PLGA | Tetraglycol and DMSO | 6 | 0.05 | 26.7 |
| **collagen | tetraglycol and acetic | 2.86 and | 0 | 54.7 |
| and PLGA | acid | 14.29 | | |
| alginate | water | 1 | 0 | 4.84 |
| alginate | water/PEG | 0.5 | 0.05 | 4.77 |
| Chitosan | 2% acetic acid in water | 0.1 | 0.1 | Unknown |
| Chitosan | 2% acetic acid in water | 0.1 | 0.001 NW | Unknown |
| Alginate | water | 0.28 | 0.1 | Unknown |
| Alginate | water | 0.28 | 0.001 NW | Unknown |

¹Solutions were measured at a shear rate of 229.45 s⁻¹ except
*at 28.68 s⁻¹ and
**at 54.36 s⁻¹.
NW refers to the inclusion of silver nanowires instead of carbon nanotubes.

Thermal inkjet printers heat a small quantity of solution to about 300° C. which vaporizes the bubble and forces nanoliter volumes of the ink through the nozzles onto the waiting substrate. We found little difficulty with nanotube aggregation due to temperature gradients or shearing of the surrounding fluid. Printed fibronectin and nanotube composites reveal that nanotube bundles are randomly oriented and uniformly dispersed.

Atomic force (AFM) and scanning electron microscopy (SEM) analysis of the printed composites reveals morphology similar to electrospun material and native vessels and also the formation of fibers in a variety of samples. For example, alginate samples have fiber structures either with or without the addition of SWNT and there appears to be no significant change in fiber morphology. However, a striking difference was observed in printed samples of collagen I when printed with polyethylene glycol (PEG) with and without SWNT. Composites of collagen hydrated with a PEG solution were found to be very globular in nature whereas a well-defined, aligned fibrous formation was observed when SWNTs dispersed using a PEG solution was added to the collagen ‘ink’. Fibrous structures were also present in PLGA sample printed with SWNT as compared to samples without. Fibers were observed in the printed PLGA samples as seen using AFM and SEM.

Inkjet printing of tissue scaffold biopolymers is possible with a wide variety of water soluble and insoluble polymers as evidenced in this work. The addition of carbon nanotubes was found to have a beneficial effect on the morphology of the printed polymers. The printed materials which form fibers upon addition of nanotubes indicates that specific structures could be printed into scaffolds; it is known that specific cell types favor certain morphologies and sizes of the structures they are seeded into. AFM comparison with decellularized blood vessel material shows that similar morphologies exist for the real tissue material and materials generated by printing nanotube/biopolymer composites.

Inkjet printing offers a viable alternative for polymer scaffold development in tissue engineering as well as for other device manufacturing needs. We have shown that not only can carbon nanotubes be printed in polymeric systems, but they generate the formation of fibers within the matrix which could be valuable in allowing cellular penetration and fluid flow into the designed scaffold. In addition, the fibrous structures that form using the inkjet printing system are similar to the surface features of real tissue. Techniques like inkjet printing allow placement of cells directly into the scaffolds to form a complete material. Our technique allows fibrous structures to form directly from the printed material without the need for added materials or coatings onto the waiting substrates, which decreases the need to manipulate the printed system. Supplementation to the properties of the scaffold by carbon nanotubes include increased strength and compressibility as shown in non-printed polymeric systems and further offer the advantage to employ the conductive nature of the SWNT for electrical stimulation of the seeded cells. Overall, we have developed new materials for use in an inkjet printing system which incorporate carbon nanotubes for their beneficial properties while also adjusting the polymer morphology toward a more preferred cell substrate.

### Materials and Methods

Hardware for our print setup is removed from a Hewlett Packard DeskJet 660c printer while the body and other components are custom-built in house. Print cartridges are prepared by first removing residual ink, sonicating the entire cartridge in water, and finally rinsing the cartridge with ethanol. The desired “inks” can then be supplied directly to the cartridges, placed in the printer, and printed onto our substrate.

Collagen I lyophilized from calf skin was used (Elastin Products Co.) with 0.05% acetic acid and magnetically stirred until completely dissolved and was then diluted to 1 mg/ml in water in accordance with previous protocols. A solution of PLGA from Purac Corp. was stirred until dissolved in 100% tetraglycol solution (Sigma Aldrich) at concentrations of 20 mg/ml and 100 mg/ml. Alternatively, 100 mg/ml PLGA was dissolved in dimethyl sulfoxide (Sigma Aldrich). Equal amounts of each PLGA solution were found best for printing. Sodium alginate (Dharma Trading Co.) solution was prepared at a concentration of 1 mg/ml and shaken until dissolved. Print preparation of 0.01% Fibronectin (Sigma Aldrich) was prepared in water. A composition of PLGA and collagen was made with the final concentrations of collagen, 2.86 mg/ml, and PLGA, 14.29 mg/ml in a 1:2.5 acetic acid to tetraglycol solvent ratio.

A solution of PLGA from Purac Corp. was stirred until dissolved in 100% tetraglycol solution (Sigma Aldrich) at concentrations of 20 mg/ml and 100 mg/ml. Alternatively, 100 mg/ml PLGA was dissolved in dimethyl sulfoxide (Sigma Aldrich). Equal amounts of each PLGA solution were found best for printing. Initially, a 10,000 MW polyethylene glycol (PEG) solution consisting of 1 g PEG, 1 mg HiPC® carbon single-wall nanotubes (Carbon Nanotechnologies, Inc.) in 10 ml water was horn sonicated (Branson) on 20% duty cycle at 40% power for ten minutes. However, upon printing of this solution we found that there was clogging. The clogging phenomenon resided from the polymer and not the tubes though. 1 ml of this solution was suspended in a 3000 MW PEG solution prepared by adding 100 mg/ml PEG in water and sonicating in a water bath for 10 minutes to obtain a uniform solution. This dispersion of nanotubes was uniform and printed repeatedly without any clogging. We refer to this solution as nanotube stock A.

Since the nanotube/PEG solutions are not compatible with PLGA as PLGA is very hydrophobic we dispersed HiPC® tubes, which are also extremely hydrophobic, in tetraglycol (Sigma Aldrich). A stock of 0.1 mg/ml HiPC® tubes in tetraglycol was sonicated with a horn sonicator on duty cycle 40% and power of 20% for ten minutes and a uniform solution was obtained. We refer to this solution as nanotube stock B.

Biopolymer/nanotube solutions were prepared using nanotube stock A with sodium alginate and collagen I. Nanotube stock B was used with PLGA and fibronectin stocks. To prepare the solutions, equal amounts of the above-described biopolymer and nanotube stocks were pipetted together and immediately printed. All solutions retained a uniform dispersion of nanotubes following mixing of the polymer and tubes. Printing of the solutions followed immediately and all solutions were printed onto clean glass slides, or copper grids for electron microscopy observation.

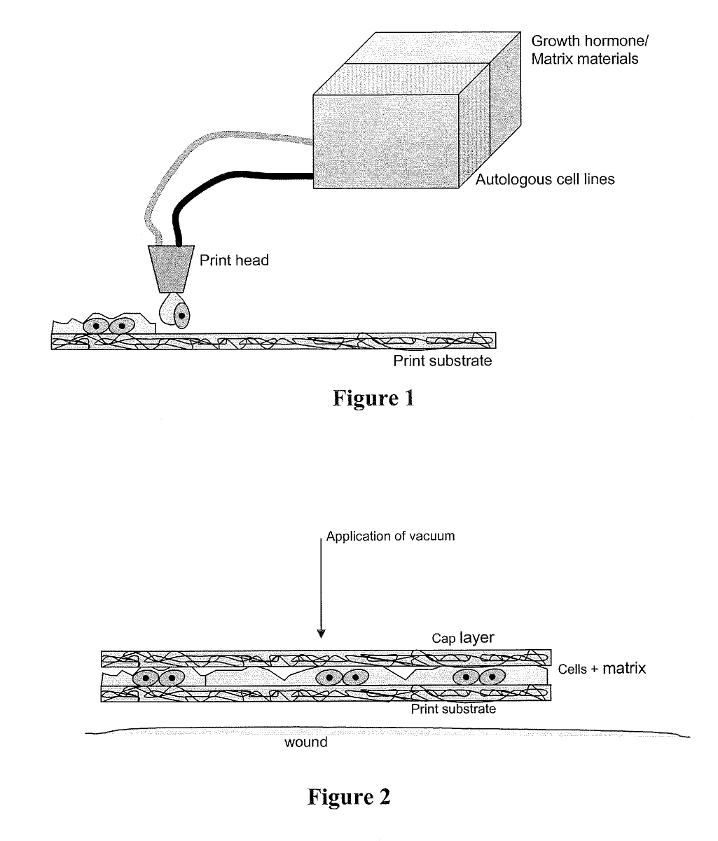

One example of the invention (schematically illustrated in FIGS. 1-2) uses an electrospun collagen or collagen-elastin or PLGA or alginate or similar scaffold embedded with protein growth factors: VEGF (Vascular Endothelial Growth Factor), Angiopoietins Ang1 and Ang2, MMP matrix metalloproteinase (MMP), FGF Fibroblast Growth Factor or fibroblast growth factor-2 (FGF2 or bFGF), DII4 (Delta-like ligand 4). On top of this is printed autologous cells harvested from the patient together with a matrix material as described above. Growth factors for the profusion of cells can be used within the concurrently printed matrix. A typical use of this scaffolded cellular material would include a top layer of electrospun collagen or collagen-nanomaterial compound on top to encase the cell matrix such that a vacuum can be applied for accelerated healing. The top layer may be removed if used without a vacuum.

### Example 2

A Hewlett Packard thermal inkjet printer model 660C was modified and used for printing of biopolymers and live human cells. Modifications to the printer include the ability to move in the two dimensions horizontally as well as positioning vertically. Standard inkjet cartridges were used. The ink was removed and cartridges cleaned by ethanol and water bath sonications.

Sodium alginate (2.5 mg/9 ml) stock concentration was prepared in deionized water. Alginate was printed directly as prepared. HipCo single-walled nanotubes (SWNT) (1 mg/ml) in a 1% Pluronic surfactant solution in water were added to the alginate stock by adding 1 ml of nanotube stock to 9 ml of alginate stock. Silver nanowires (NW) were prepared according to published methods. The concentration of nanowires is unknown although it is estimated to be about 10 ug/ml. One ml of the NW stock was added to 9 ml of alginate stock to prepare printable solutions. All polymer solutions of alginate were printed at 5, 10 or 15 printed passes to develop sufficient substrates for cell seeding. Following drying of the printed alginate the biopolymer was cross-linked with 50 mM CaCl solution for 10 minutes followed by washing with 1% NaCl to end the cross-linking process. Slides with cross-linked alginate were sterilized by soaking in 70% ethanol solution for 10 minutes followed by rinses with sterile phosphate buffered saline (PBS).

Chitosan solutions were also printed consisting of a 1% aqueous chitosan in 2% acetic acid, diluted in deionized water to a printable concentration of 0.1% chitosan. The same SWNT or NW stocks were used as in section 2. However, 30 printed passes of this polymer were used for cell seeding. Chitosan printed onto glass slides was cross-linked by treatment with ultraviolet light overnight. The samples were then sterilized as described in part 2.

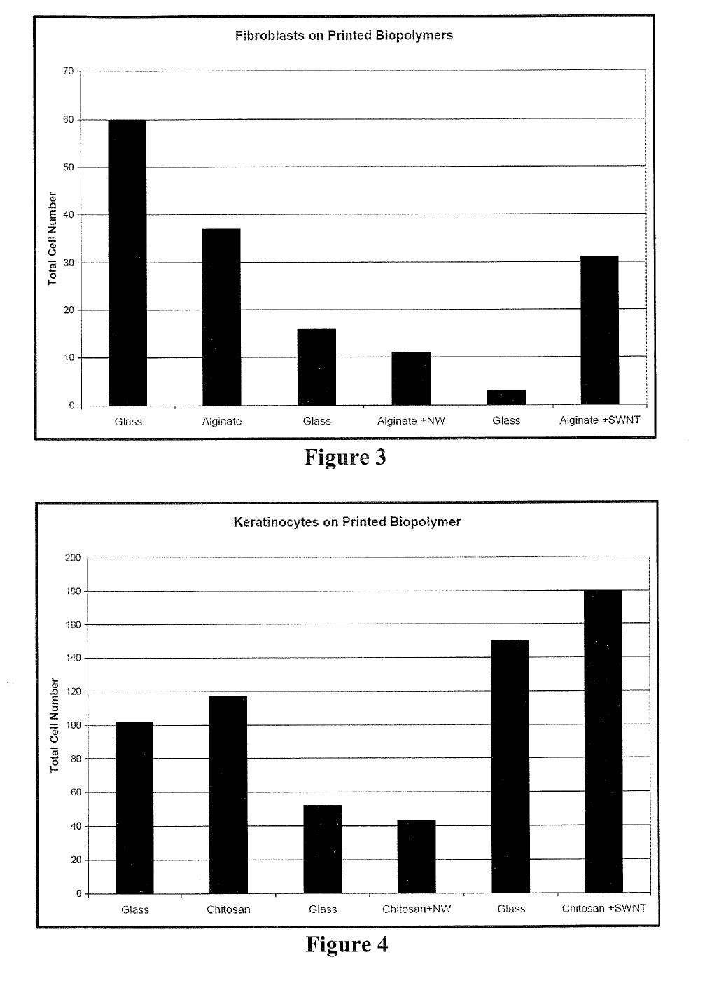

Primary human fibroblasts or keratinocytes from clinical patient samples were applied in media suspension to sterilized slides with printed biopolymers. Cells were allowed to proliferate for 24 hr. prior to fixation in methanol and staining with eosin. Printed alginate was stained with alcian blue dye and printed chitosan stained with eosin to indicate areas of printed material or the glass substrate. Data are given in FIGS. 3-4.

Slides of the printed material and cultured cells were examined under light microscopy to determine the number of cells adherent on the glass only areas compared to areas of printed biopolymer. The figure demonstrates that cells were maintained and viable on the printed materials. No printed materials, including those containing silver nanoparticles or SWNT were toxic or reduced cell adhesion and growth. The printed materials were compatible with growth of keratinocytes.

Human colorectal epithelial cells (HCT 116 line) were printed in PBS at a concentration of five million cells per ml. Human primary fibroblasts were printed in PBS at a concentration of 330,000/ml. Both cell types were printed directly into cell culture media and allowed to proliferate for six days. Live cell populations were analyzed using calcein fluorescent staining in PBS.

### REFERENCE LIST

1. Mironov, V., Boland, T., Trusk, T., Forgacs, G. & Markwald, R. R. Organ printing: computer-aided jet-based 3D tissue engineering. Trends in Biotechnology 21, 157-161 (2003).
2. MacDonald, R. A., Laurenzi, B. F., Viswanathan, G., Ajayan, P. M. & Stegemann, J. P. Collagen-carbon nanotube composite materials as scaffolds in tissue engineering. Journal of Biomedical Materials Research Part A 74A, 489-496 (2005).
3. Webster, T. J., Waid, M. C., McKenzie, J. L., Price, R. L. & Ejiofor, J. U. Nano-biotechnology: carbon nanofibres as improved neural and orthopaedic implants. Nanotechnology 15, 48-54 (2004).
4. Correa-Duarte, M. A. et al. Fabrication and biocompatibility of carbon nanotube-based 3D networks as scaffolds for cell seeding and growth. Nano Letters 4, 2233-2236 (2004).
5. Marrs, B., Andrews, R., Rantell, T. & Pienkowski, D. Augmentation of acrylic bone cement with multiwall carbon nanotubes. Journal of Biomedical Materials Research Part A 77A, 269-276 (2006).
6. Dersch, R., Steinhart, M., Boudriot, U., Greiner, A. & Wendorff J. H. Nanoprocessing of polymers: applications in medicine, sensors, catalysis, photonics. Polymers for Advanced Technologies 16, 276-282 (2005).
7. Zhang, S. G., Marini, D. M., Hwang, W. & Santoso, S. Design of nanostructured biological materials through self-assembly of peptides and proteins. Current Opinion in Chemical Biology 6, 865-871 (2002).
8. Zanello, L. P., Zhao, B., Hu, H. & Haddon, R. C. Bone cell proliferation on carbon nanotubes. Nano Letters 6, 562-567 (2006).
9. Wang, Y. C. & Ho, C. C. Micropatterning of proteins and mammalian cells on biomaterials. Faseb Journal 18, (2004).
10. Yoon, J. J. & Park, T. G. Degradation behaviors of biodegradable macroporous scaffolds prepared by gas foaming of effervescent salts. Journal of Biomedical Materials Research 55, 401-408 (2001).
11. Balakrishnan, B. & Jayakrishnan, A. Self-cross-linking biopolymers as injectable in situ forming biodegradable scaffolds. Biomaterials 26, 3941-3951 (2005).
12. Xu, F. L., Li, Y. B., Han, J. M. & Lv, G. Y. Biodegradable porous nano-hydroxyapatite/alginate scaffold. Eco-Materials Processing & Design Vi 486-487, 189-192 (2005).
13. Boland, E. D. et al. Electrospinning collagen and elastin: Preliminary vascular tissue engineering. Frontiers in Bioscience 9, 1422-1432 (2004).
14. Stitzel, J. et al. Controlled fabrication of a biological vascular substitute. Biomaterials 27, 1088-1094 (2006).
15. Burg, K. J. L. & Boland, T. Minimally invasive tissue engineering composites and cell printing. Ieee Engineering in Medicine and Biology Magazine 22, 84-91 (2003).
16. Nakamura, M. et al. Biocompatible inkjet printing technique for designed seeding of individual living cells. Tissue Engineering 11, 1658-1666 (2005).
17. Varghese, D. et al. Advances in tissue engineering: Cell printing. Journal of Thoracic and Cardiovascular Surgery 129, 470-472 (2005).
18. Xu, T., Jin, J., Gregory, C., Hickman, J. J. J. & Boland, T. Inkjet printing of viable mammalian cells. Biomaterials 26, 93-99 (2005).
19. Khang, D., Sato, M. & Webster, T. J. Directed osteoblast functions on micro-aligned patterns of carbon nanofibers on a polymer matrix. Reviews on Advanced Materials Science 10, 205-208 (2005).
20. Bianco, A. & Prato, M. Can carbon nanotubes be considered useful tools for biological applications? Advanced Materials 15, 1765-1768 (2003).
21. Chlopek, J. et al. In vitro studies of carbon nanotubes biocompatibility. Carbon 44, 1106-1111 (2006).
22. Jie, M. et al. Blood coagulation resistance of nonwoven single-walled carbon nanotubes and its implications for implantable prostheses. New Carbon Materials 19, 166-171 (2004).
23. Sato, Y. et al. Influence of length on cytotoxicity of multi-walled carbon nanotubes against human acute monocytic leukemia cell line THP-I in vitro and subcutaneous tissue of rats in vivo. Molecular Biosystems 1, 176-182 (2005).
24. Shi, X. F. et al. Rheological behaviour and mechanical characterization of injectable poly(propylene fumarate)/single-walled carbon nanotube composites for bone tissue engineering. Nanotechnology 16, S531-S538 (2005).
25. Xu, T., Jin, J., Gregory, C., Hickman, J. J. J. & Boland, T. Inkjet printing of viable mammalian cells. Biomaterials 26, 93-99 (2005).
26. Xu, T., Jin, J., Gregory, C., Hickman, J. J. J. & Boland, T. Inkjet printing of viable mammalian cells. Biomaterials 26, 93-99 (2005).

The foregoing is illustrative of the present invention, and is not to be construed as limiting thereof. The invention is defined by the following claims, with equivalents of the claims to be included therein.

That which is claimed is:

1. A method of carrying out an autologous tissue implant in a subject in need thereof, comprising the steps of:
(a) forming an autologous tissue implant from autologous cells collected from a subject by, in any order or in combination, (i) ink-jet printing said cells on a substrate and (ii) inkjet printing a scaffold for said cells on said substrate, said scaffold comprising nanoparticles and a physiologically acceptable polymer, and (iii) optionally repeating steps (i) and (ii) to form said autologous tissue implant; and then
(b) implanting said autologous tissue implant in said subject.

2. The method of claim 1, further comprising the step of:
applying a preformed or ink-jet printed cap layer to said implant after said forming step.

3. The method of claim 1, further comprising:
repeating step (a) from 1 to 1000 times.

4. The method of claim 1, wherein said ink jet printing is carried out on an electrospun or electrosprayed substrate

5. The method of claim 1, wherein said nanoparticles are antibacterial nanoparticles.

6. The method of claim 1, wherein said nanoparticles are metal nanoparticles.

7. The method of claim 1, wherein said nanoparticles are electrically conductive.

8. The method of claim 1, wherein said nanoparticles are silver nanoparticles.

9. The method of claim 1, wherein said autologous cells comprise skin cells, said subject is afflicted with a wound, and said autologous tissue implant is applied to said wound, optionally followed by treating said wound, said autologous tissue implant, or both said wound and said autologous tissue implant with negative pressure wound therapy.

10. The method of claim 9, wherein said wound is a burn.

11. The method of claim 1, wherein said autologous cells comprise smooth muscle cells or endothelial cells, said subject is afflicted with a defective region in a smooth muscle organ wall, and said autologous tissue implant is applied to said defective region.

12. The method of claim 1, wherein said autologous cells are cardiac muscle cells, said subject is afflicted with a defective region in a heart wall, and said autologous tissue implant is applied to said defective region.

13. The method of claim 1, wherein said autologous cells are chondrocytes, said subject is afflicted with a defective region in cartilage, and said autologous tissue implant is applied to said defective region.

14. The method of claim 1, wherein said autologous cells are fat cells, said subject has a region in need of tissue augmentation, and said autologous tissue implant is implanted into said region in need of tissue augmentation.

15. The method of claim 1, wherein said autologous cells comprise skin and fat cells, said subject is afflicted with a wound in need of tissue augmentation, and said autologous tissue implant is applied to said wound, optionally followed by treating said wound, said autologous tissue implant, or both said wound and said autologous tissue implant with negative pressure wound therapy.

16. The method of claim 1, wherein said step of ink jet printing a scaffold is carried out by printing a composition comprising nanoparticles, a polymer and a solvent.

17. The method of claim 16, wherein said polymer comprises polylactide or a copolymer thereof, and wherein said solvent comprises tetraglycol and DMSO.

18. The method of claim 16, wherein said polymer comprises collagen, and wherein said solvent comprises water and an acid (e.g., acetic acid, citric acid, and/or HCl).

19. The method of claim 16, wherein said polymer comprises collagen and polycitrate, and wherein said solvent comprises 1,4-dioxane.

20. The method of claim 16, wherein said composition further comprises polyethylene glycol, said polyethylene glycol having a molecular weight not greater than 8,000 daltons.

21. The method of claim 16 wherein said nanoparticles comprise nanotubes.

22. The method of claim 16, wherein said scaffold is patterned.

23. An autologous tissue implant produced by the process of claim 1.
